(19) United States
(12) Patent Application Publication
(10) Pub. No.: US 2024/0363250 A1
(43) Pub. Date: Oct. 31, 2024

(54) ARTIFICIAL INTELLIGNECE-BASED HEMODIALYSIS DATA PROCESSING METHOD AND SYSTEM

(71) Applicant: Sanghyuk KWAK, Daejeon (KR)

(72) Inventor: Sanghyuk KWAK, Daejeon (KR)

(21) Appl. No.: 18/764,877

(22) Filed: Jul. 5, 2024

Related U.S. Application Data
(63) Continuation of application No. PCT/KR2023/000065, filed on Jan. 3, 2023.

(30) Foreign Application Priority Data
Jan. 7, 2022 (KR) 10-2022-0002582
Dec. 21, 2022 (KR) 10-2022-0180184

(51) Int. Cl. G16H 50/20; G16H 10/60; G16H 50/30

(52) U.S. Cl. CPC G16H 50/20; G16H 10/60; G16H 50/30

(57) ABSTRACT

The present disclosure relates to an artificial intelligence-based hemodialysis data processing method, device, and system. The method of the present disclosure may comprise the steps of: extracting pre-stored identification information of a patient; acquiring health status information measured for the patient; mapping the extracted identification information and the acquired health status information; acquiring information of the amount of blood required for dialysis for that day, calculated for the patient on the basis of the mapped identification information and health status information; and outputting the acquired information of the amount of blood required for dialysis for that day.

### CROSS-REFERENCE TO RELATED APPLICATIONS

The present application is a continuation of International Patent Application No. PCT/KR2023/000065, filed on Jan. 3, 2023, which is based upon and claims the benefit of priority to Korean Patent Application Nos. 10-2022-0002582 filed on Jan. 7, 2022 and 10-2022-0180184 filed on Dec. 21, 2022. The disclosures of the above-listed applications are hereby incorporated by reference herein in their entirety.

### BACKGROUND

Embodiments of the present disclosure described herein relate to hemodialysis data processing, and more particularly, relate to artificial intelligence (AI)-based hemodialysis data processing method, device, and system.

Hemodialysis refers to a treatment method that requires more than 4 hours at a time for a patient with end-stage chronic renal failure and needs to be continued three times a week for life.

During the hemodialysis, it is necessary to continuously monitor and record data of the patient's vital signs and sensor data of a hemodialysis device.

However, the hemodialysis device transmits data in a medical data exchange standard file format (i.e., HL7 format). The file format thus transmitted is different from an electronic medical record system used by medical staff, and thus it is difficult for humans to read and interpret the file format.

Moreover, it is also necessary to continuously record the patient's nursing and treatment records during hemodialysis. The medical staff needs to use a hemodialysis nursing record written by hand on a paper or needs to enter records directly into the nursing record screen of the electronic medical record software on a computer.

However, during this process, there is a possibility that patient data is stored differently from actual information due to information errors or omissions. This may lead to major medical errors in later treatment, and thus it's important to be prepared.

### SUMMARY

Embodiments of the present disclosure provide a hemodialysis data processing method that may provide customized hemodialysis information based on AI and monitors a hemodialysis process based on AI, and a system therefor.

Problems to be solved by the present disclosure are not limited to the problems mentioned above, and other problems not mentioned will be clearly understood by those skilled in the art from the following description.

According to an embodiment, an artificial intelligence (AI)-based hemodialysis data processing method includes extracting pre-stored identification information for a target patient, acquiring health status information measured for the target patient, mapping the extracted identification information and the acquired health status information, acquiring recommended same-day hemodialysis demand information calculated for the target patient based on the mapped identification information and the mapped health status information, and outputting the acquired recommended same-day hemodialysis demand information.

In this case, the acquiring of the recommended same-day hemodialysis demand information may include predicting dry weight data based on change data of a weight and blood pressure of the target patient during a predetermined period by using an AI model, correcting the predicted dry weight data by reflecting a result trained by labeling a standard dry weight based on a type, a dose, and duration of medication that the target patient is taking, and calculating the recommended same-day hemodialysis demand based on the corrected dry weight data. In the calculating of the recommended same-day hemodialysis demand, when an event in which a health status of the target patient changes occurs in a previous hemodialysis process of the target patient, and a hemodialysis amount of the target patient is adjusted depending on the occurred event, data regarding the adjusted hemodialysis amount may be finally calculated by correcting the recommended same-day hemodialysis demand based on the trained result.

Moreover, the method according to an embodiment of the present disclosure may further include monitoring a hemodialysis process of the target patient depending on the recommended same-day hemodialysis demand information, and acquiring vital sign data during hemodialysis of the target patient in the monitoring. The vital sign data may include respective sequential data that comes out when a blood flow of the target patient passes through a hemodialysis device.

Furthermore, the method according to an embodiment of the present disclosure may further include performing training by labeling a clinical event in the respective sequential data by using an AI model.

Besides, the method according to an embodiment of the present disclosure may further include obtaining data obtained by predicting a vital sign according to the hemodialysis of the target patient based on the acquired vital sign data, generating clinical event data according to the hemodialysis of the target patient based on the predicted data, and outputting the generated clinical event data.

Also, the method according to an embodiment of the present disclosure may further include determining whether there is a health anomaly according to the hemodialysis of the target patient based on the acquired vital sign data, clinical event data, and unique characteristic information related to hemodialysis, and causing the determination result of whether there is the health anomaly to be output.

In addition, the method according to an embodiment of the present disclosure may further include performing training by labeling sequential data at a point in time, when the health anomaly occurs, and diagnosis and response content of a medical institution for resolving the health anomaly by using an AI model. The determination result of whether there is the health anomaly may include the diagnosis and the response content of the medical institution according to the trained result of the AI model.

According to an embodiment, an AI-based hemodialysis data processing system includes at least one terminal, and a server including a processor that performs data communication with the terminal. The processor extracts pre-stored identification information for a target patient, acquires health status information measured for the target patient, maps the extracted identification information and the acquired health status information, acquires recommended same-day hemodialysis demand information calculated for the target patient based on the mapped identification information and the mapped health status information, and causes the acquired recommended same-day hemodialysis demand information to be output.

In this case, when acquiring the recommended same-day hemodialysis demand information, the processor predicts dry weight data based on change data of a weight and blood pressure of the target patient during a predetermined period by using an AI model, corrects the predicted dry weight data by reflecting a result trained by labeling a standard dry weight based on a type, a dose, and duration of medication that the target patient is taking, calculates the recommended same-day hemodialysis demand based on the corrected dry weight data, and finally calculates data regarding the adjusted hemodialysis amount by correcting the recommended same-day hemodialysis demand based on the trained result when an event in which a health status of the target patient changes occurs in a previous hemodialysis process of the target patient, and a hemodialysis amount of the target patient is adjusted depending on the occurred event.

Moreover, the processor may monitor a hemodialysis process of the target patient depending on the recommended same-day hemodialysis demand information, and acquires vital sign data during hemodialysis of the target patient in the monitoring. The vital sign data may include respective sequential data that comes out when a blood flow of the target patient passes through a hemodialysis device.

Furthermore, the processor may perform training by labeling a clinical event in the respective sequential data by using an AI model.

Besides, the processor may obtain data obtained by predicting a vital sign according to the hemodialysis of the target patient based on the acquired vital sign data, may generate clinical event data according to the hemodialysis of the target patient based on the predicted data, and may cause the generated clinical event data to be output.

Also, the processor may determine whether there is a health anomaly according to the hemodialysis of the target patient based on the acquired vital sign data, clinical event data, and unique characteristic information related to hemodialysis, and may cause the determination result of whether there is the health anomaly to be output.

In addition, the processor may perform training by labeling sequential data at a point in time, when the health anomaly occurs, and diagnosis and response content of a medical institution for resolving the health anomaly by using an AI model. The determination result of whether there is the health anomaly may include the diagnosis and the response content of the medical institution according to the trained result of the AI model.

Besides, a computer program stored in a computer-readable recording medium for execution to implement the present disclosure may be further provided.

In addition, a computer-readable recording medium for recording a computer program for performing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram illustrating an AI-based hemodialysis data processing system, according to an embodiment of the present disclosure;

FIG. 2 is a block diagram of the server of FIG. 1;

FIG. 3 is a block diagram of the processor of FIG. 2;

FIG. 4 is a diagram for describing a processing process in an AI-based hemodialysis data processing system, according to an embodiment of the present disclosure; and

FIG. 5 is a diagram for describing an AI-based hemodialysis data processing method, according to an embodiment of the present disclosure.

### DETAILED DESCRIPTION

The above and other aspects, features and advantages of the present disclosure will become apparent from the following description of the following embodiments given in conjunction with the accompanying drawings. The present disclosure, however, may be embodied in various different forms, and should not be construed as being limited only to the illustrated embodiments. Rather, these embodiments are provided as examples so that the present disclosure will be thorough and complete, and will fully convey the scope of the present disclosure to those skilled in the art. The present disclosure may be defined by the scope of the claims.

The terms used herein are provided to describe embodiments, not intended to limit the present disclosure. In the specification, the singular forms include plural forms unless particularly mentioned. The terms “comprises” and/or “comprising” used herein do not exclude the presence or addition of one or more other components, in addition to the aforementioned components. The same reference numerals denote the same components throughout the specification. As used herein, the term “and/or” includes each of the associated components and all combinations of one or more of the associated components. It will be understood that, although the terms “first”, “second”, etc., may be used herein to describe various components, these components should not be limited by these terms. These terms are only used to distinguish one component from another component. Thus, a first component that is discussed below could be termed a second component without departing from the technical idea of the present disclosure.

Unless otherwise defined, all terms (including technical and scientific terms) used herein have the same meaning as commonly understood by those skilled in the art to which the present disclosure pertains. The terms, such as those defined in commonly used dictionaries, should not be interpreted in an idealized or overly formal sense unless expressly so defined herein.

Hereinafter, an embodiment of the present disclosure will be described in detail with reference to the accompanying drawings.

In this specification, an AI-based hemodialysis data processing device according to an embodiment of the present disclosure includes all various devices capable of providing results to a user by performing arithmetic processing. For example, a data processing device according to an embodiment of the present disclosure may include all of at least one computer or computing device, a server device, and a portable terminal, or may be in any one form.

Here, for example, the computer may include a notebook computer, a desktop computer, a laptop computer, a tablet PC, a slate PC, and the like, which are equipped with a web browser.

The server device may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet terminal (Wibro) terminal, and a wearable device such as a timepiece, a ring, a bracelet, an anklet, a necklace, glasses, a contact lens, or a head-mounted device (HMD).

In this specification, an information provision control model may be defined or a related platform may be built in relation to the AI-based hemodialysis data processing device according to an embodiment of the present disclosure. It may be created and provided by a computer based on big data and AI technology, and may be implemented by using or referencing information and communication technology (ICT) such as extended Reality, which collectively refers to virtual reality, augmented reality, and mixed reality, blockchain technology for the security of personal information of users using information provision devices, and the like. However, in this specification, detailed descriptions of such the ICT technology will be omitted with reference to known technologies.

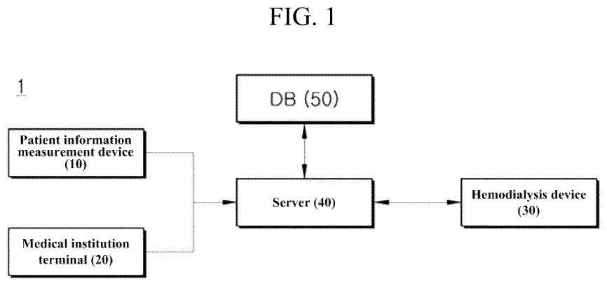

FIG. 1 is a diagram illustrating an AI-based hemodialysis data processing system 1, according to an embodiment of the present disclosure.

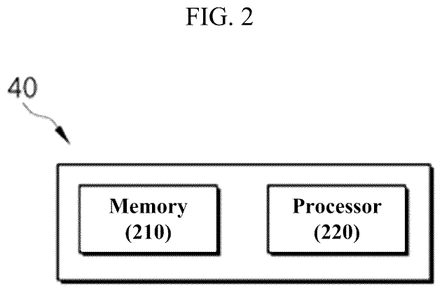

FIG. 2 is a block diagram of the server of FIG. 1.

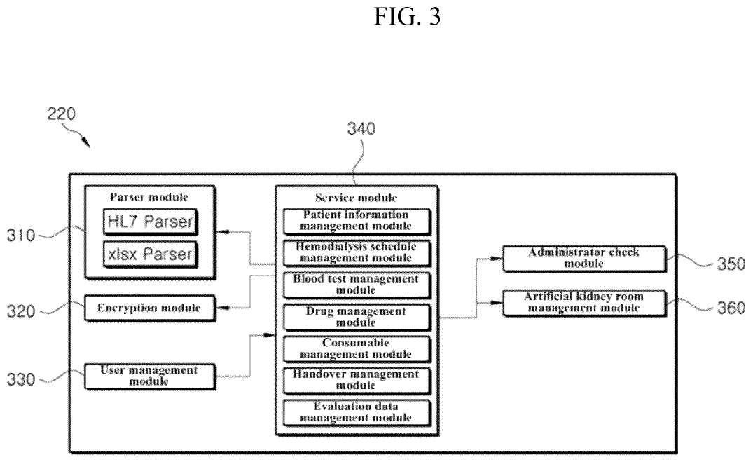

FIG. 3 is a block diagram of the processor of FIG. 2.

A system that provides an AI-based hemodialysis data processing service according to an embodiment of the present disclosure may include a patient information measurement device 10, a medical institution terminal 20, a hemodialysis device 30, and a server 40. At this time, the AI-based hemodialysis data processing system 1 may also include a DB 50 that stores data by communicating with the server 40.

According to an embodiment, the AI-based hemodialysis data processing system 1 may be implemented such that one or more components are added in relation to performing an operation according to an embodiment of the present disclosure, in addition to the components shown in FIG. 1.

The AI-based hemodialysis data processing process according to an embodiment of the present disclosure may use an application provided by the server 40 or information provided in a form of a web service through the web.

For example, the application may be provided by the server 40, may be downloaded and installed by the medical institution terminal 20 or the hemodialysis device 30. When the application is executed, the application may provide a related service user interface (UI) to receive information about a target patient or may provide service information related to various hemodialysis services related to the present disclosure. In this case, the medical institution terminal 20 or the hemodialysis device 30 may output information related to the target patient or the target patient's hemodialysis to a display (or augmented reality method) on an application execution screen through the server 40.

In relation to this, the server 40 may provide the patient information measurement device 10, the medical institution terminal 20, and the hemodialysis device 30 with an algorithm or logic for processing AI-based hemodialysis data according to an embodiment of the present disclosure or/and application programming interface (API) or plug-in related thereto.

In the meantime, the server 40 may build and provide a service platform for providing an AI-based hemodialysis data processing service and may provide the service so as to fetch patient information measured from the patient information measurement device 10 through the service platform or to receive or print information from the medical institution terminal 20 or the hemodialysis device 30.

The patient information measurement device 10 may measure the patient information such as a patient's weight and blood pressure for dialysis for that day. The patient information measurement device 10 may include various sensor devices such as a scale and a camera.

The patient information measurement device 10 may directly transmit the patient information measured for the target patient to the medical institution terminal 20.

Alternatively, the patient information measurement device 10 transmits the patient information measured for the target patient to the server 40, and the server 40 may transmit the patient information to the corresponding medical institution terminal 20.

In the process, at least one of the patient information measurement device 10 and the server 40 may map the measured patient information and the target patient and may transmit the mapped information.

The medical institution terminal 20 may calculate a dry weight and a recommended same-day hemodialysis demand of the target patient based on the patient information of the target patient measured by the patient information measurement device 10.

The medical institution terminal 20 may output information about the target patient's dry weight and recommended same-day hemodialysis demand calculated in this way to a screen, and may also transmit the information to the server 40.

The dry weight may refer to the patient's appropriate weight at which blood pressure remains normal without edema. In other words, the dry weight may indicate the patient's normal target weight to be reached by removing moisture from the body through same-day hemodialysis. However, the dry weight may be individually set as a same-day dry weight value according to the opinion of a doctor diagnosing the target patient's status.

The medical institution terminal 20 may be a fixed terminal such as a PC, a monitor, or a digital signage, or a mobile terminal such as a smartphone, a tablet PC, or a laptop. Alternatively, the medical institution terminal 20 may be in a form of a wearable device such as a smart watch or a head-mounted display (HMD). Alternatively, the medical institution terminal 20 may be a dedicated device for medical use or AI-based hemodialysis data processing service according to an embodiment of the present disclosure, or may be a device equipped with software.

The hemodialysis device 30 may store the recommended same-day hemodialysis demand information of the target patient transmitted from the server 40 in the DB and may output the stored information through the display.

The hemodialysis device 30 may include various sensors to transmit various pieces of data, which are collected from the target patient during the hemodialysis process, to the server 40.

Each component constituting the AI-based hemodialysis data processing system 1 of FIG. 1 may include a communication module for data communication with other components. For example, these communication modules may include at least one of a wired communication module, a wireless communication module, a short-range communication module, and a location information module.

Here, in addition to various wired communication modules such as a Local Area Network (LAN) module, a Wide Area Network (WAN) module, or a Value Added Network (VAN) module, the wired communication module may include a variety of cable communication modules such as Universal Serial Bus (USB), High Definition Multimedia Interface (HDMI), Digital Visual Interface (DVI), recommended standard-232 (RS-232), power line communication, or plain old telephone service (POTS).

The wireless communication module may include a wireless communication module for supporting various wireless communication methods such as Global System for Mobile (GSM) communication, Code Division Multiple Access (CDMA), Wideband Code Division Multiple Access (WCDMA), Universal Mobile Telecommunication System (UMTS), Time Division Multiple Access (TDMA), Long Term Evolution (LTE), 4G, 5G, and 6G in addition to a Wi-Fi module and a wireless broadband module.

The short-range communication module may be used for short range communication, and may support short-range communication by using at least one of Bluetooth™, radio frequency identification (RFID), infrared data association (IrDA), ultra wideband (UWB), ZigBee, near field communication (NFC), Wireless-Fidelity (Wi-Fi), Wi-Fi Direct, and wireless universal serial bus (Wireless USB) technologies.

For example, the location information module may be a module for obtaining a location (or a current location) of the medical institution terminal 20 and may typically include a global positioning system (GPS) module or a Wireless Fidelity (Wi-Fi) module. For example, when utilizing the GPS module, the location information module may obtain the location of the medical institution terminal 20 by using a signal received from a GPS satellite. For another example, when utilizing the Wi-Fi module, the location information module may obtain the location of the medical institution terminal 20 based on information of a wireless access point (AP) exchanging wireless signals with the Wi-Fi module. As needed, the location information module may perform one of functions of the other modules of the communication module to obtain data regarding the location of the medical institution terminal 20 alternatively or additionally. The location information module is a module used to obtain the location (or current location) of the medical institution terminal 20, but is not limited to a module that directly calculates or obtains the location of the medical institution terminal 20. The location information module may be built into the medical institution terminal 20 to provide location information of the medical institution terminal 20 to the server 40.

The server 40 will be described with reference to FIG. 2.

The server 40 may include a memory 210 and at least one processor 220 communicating with the memory 210.

In this case, the memory 210 of FIG. 2 may be the DB 50 of FIG. 1 described above or a separate storage medium in the form of a cloud. In the meantime, the number of the memory 210 is not necessarily one.

The processor 220 may perform data communication and control operations on components of the AI-based hemodialysis data processing system 1 according to an embodiment of the present disclosure shown in FIG. 1.

The processor 220 may perform or control various operations, processing, data configuration, and provision on a service platform interacting with other components. The processor 220 may store data regarding various algorithms available in the process in the memory 210 or programs that reproduce the algorithms, and may perform various operations for providing an AI-based hemodialysis data processing service according to an embodiment of the present disclosure by using data stored in the memory 210.

The processor 220 may create and train at least one or more learning models related to the AI-based hemodialysis data processing service and may be used to provide an AI-based hemodialysis data processing service according to an embodiment of the present disclosure by inputting user information. Big data and AI technologies may be used in the training process. Besides, the processor 220 may also process sensitive personal information, disease information, or the like in the target patient's information by using a blockchain technology, as needed. Moreover, related information according to an embodiment of the present disclosure may be used through various ICT technologies such as Internet of Things (IoT), extended Reality (XR), and the like.

An operation of the processor 220 will be described with reference to FIG. 3.

Referring to FIG. 3, the processor 220 may include a parser module 310, an encryption module 320, a user management module 330, a service module 340, an administrator check module 350, an artificial kidney room management module 360, and the like.

However, the present disclosure is not limited thereto, and some of the modules shown in FIG. 3 may be merged to form one, or vice versa.

The parser module 310 may include a HL7 parser and an xlsx parser.

The HL7 parser may read HL7 format files, which are generated by the hemodialysis device 30, from a memory. The HL7 parser may determine whether the read file complies with the HL7 Version 2 file standard (determining whether a specific string is present in the first line of a file) and may extract data and attributes from the files read from the memory. Afterwards, the HL7 parser may encrypt data by using the encryption module 320 and may transmit the encrypted data to a patient information management module of the service module 340.

The xlsx parser may read Excel format files downloaded from the website of an external inspection agency from the memory and may determine whether the read Excel files comply with a blood test result format. The xlsx parser may extract data and attributes from the files read from the memory, may encrypt data by using the encryption module 320, and may transmit the encrypted data to the patient information management module of the service module 340.

The encryption module 320 may select an encryption method. The encryption method may include encryption methods such as SHA256, SHA512, and SEED. The encryption module 320 may create/save/read/delete an encryption key according to the selected encryption method and may encrypt and decrypt data according to the selected encryption method.

The user management module 330 may change a user's login password, and may store the user's access records (connection time, logout time, IP address, MAC address, or the like).

The service module 340 may include at least one or more of a patient information management module, a hemodialysis schedule management module, a blood test management module, a drug management module, a consumable management module, a handover management module, and an evaluation data management module.

First of all, the patient information management module may encrypt/decrypt the target patient's personal information by using the encryption module 320. The patient information management module may manage the target patient's personal information and may communicate with a public institution server (e.g., National Health Insurance Corporation) to query information about infectious disease/overseas disease.

The hemodialysis schedule management module may perform the function of connecting a hemodialysis patient and a bed and the function of reserving/changing/deleting the hemodialysis schedule and time of the hemodialysis patient.

The blood test management module may automatically generate a scheduled test date according to the test cycle (e.g., 1 month, 3 months, 6 months, 12 months, or the like) for the target patient's blood test, and may perform the function of automatically determining the test result (e.g., when the test result is outside the already set normal range) and the function (e.g., re-test after 2 days, 2 weeks, 1 month, or the like) of recommending follow-up tests to medical staff. The blood test management module may classify stored blood test results into a normal result and an abnormal result and then may generate a report.

The drug management module may perform the function of accumulating and managing the drugs prescribed to a patient depending on the dosage and period, and may perform the function of visualizing (e.g., the dosage for each drug with time) drugs and dosages for each ingredient based on the drug ingredient standard code, and simultaneously linking and visualizing the severity of symptoms or test results preset by the user.

The consumable management module may perform a function of registering/changing/deleting a consumable list, may manage the purchase/use/damage/deterioration of consumables related to hemodialysis, may perform the function of setting the minimum reserve amount of consumables, and may report a purchase list when the consumables decrease to be smaller than or equal to minimum reserves.

The handover management module may create/edit/delete an author, a recipient, a target patient, and handover details. The recipient may confirm and record that the handover has been received.

Besides, the evaluation data management module may manage evaluation data.

The administrator check module 350 may register/edit/delete users, may set the use scope of a program for each user, may print a user's access history report, and may reset the user's login password.

The artificial kidney room management module 360 may register/change/delete information about a hemodialysis device of an artificial kidney room and may register/change/delete the operating hours and treatment schedule of the artificial kidney room.

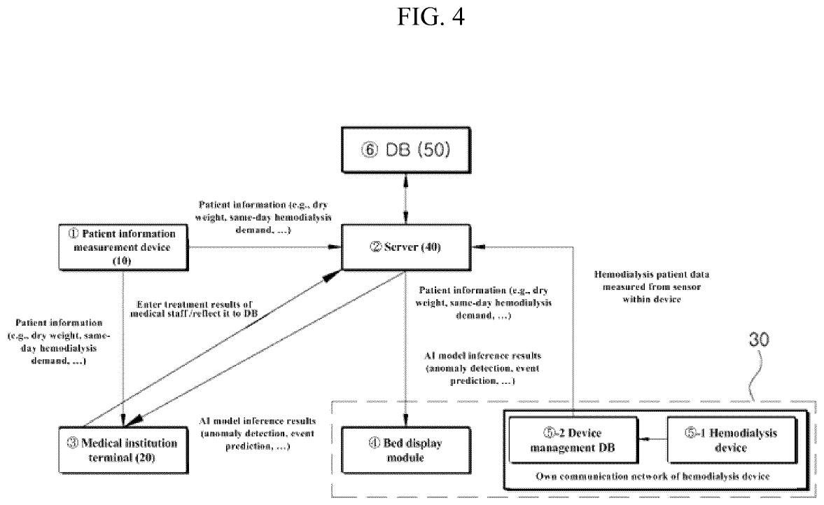

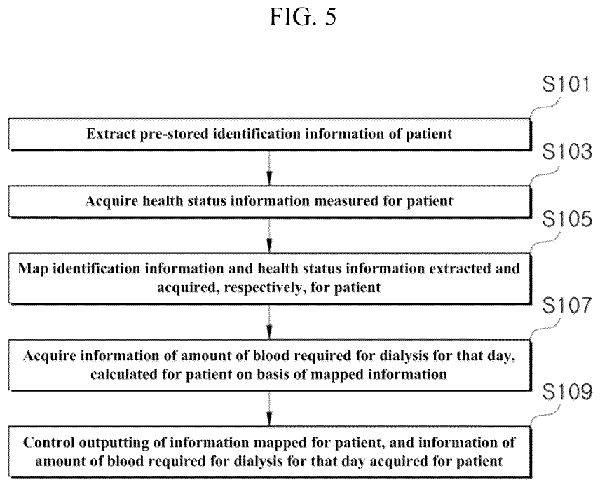

FIGS. 4 and 5 are diagrams for describing an AI-based hemodialysis data processing process, according to an embodiment of the present disclosure.

FIG. 4 describes an entire processing process within the AI-based hemodialysis data processing system 1.

In accordance with the AI-based hemodialysis data processing system 1 according to an embodiment of the present disclosure, target patient basic information, patient disease information, and bed reservation information may be collectively managed. In this case, methods such as login, facial recognition, fingerprint recognition, QR code recognition, and NFC may be used. In the meantime, the AI-based hemodialysis data processing system 1 according to an embodiment of the present disclosure may monitor a patient's vital signs during a hemodialysis process, may detect an anomaly based on an AI model, and may quickly respond thereto.

Referring to FIG. 4, first of all, the patient information measurement device 10 and the hemodialysis device 40 may be linked to each other.

As mentioned above, the patient information measurement device 10 may identify target patient information through facial recognition of a patient or QR code recognition, may read the identified target patient information from the DB 50 through the server 40, and may collectively manage the identified target patient information during examination/bed movement. Here, the bed is one of the components of the hemodialysis device 30 and refers to the part for hemodialysis of the patient.

In this disclosure, only the target patient who visits on a pre-designated date may be applied for recognition such as facial recognition with reference to the target patient's schedule information, not simply recognizing the target patient. In other words, there may be a procedure of verifying whether a visitor (patient) is assigned to a current visitor session before face recognition.

The patient information measurement device 10 may measure the patient's health status information, such as a weight and blood pressure, on the day of the patient's hemodialysis and may map the measured health status information into the identified patient information. The mapped information may be delivered to the medical institution terminal 20 (or via the server 40).

Accordingly, the recommended same-day hemodialysis demand based on dry weight of the target patient may be calculated on the basis of the information received from the medical institution terminal 20. The dry weight-based recommended same-day hemodialysis demand calculated in this way may be transmitted to the server 40 and may be delivered to the hemodialysis device 30.

The hemodialysis device 30 may be a display module (i.e., a display placed near a bed/the hemodialysis device 30), and may display the hemodialysis demand and patient data of the patient of the corresponding bed. The patient bed may be automatically assigned based on a rule by the server 40 receiving patient data from the patient information measurement device 10 in consideration of whether the disease status of a target patient visiting a ward, a bed preparation time (due to disinfection, cleaning, etc.), hemodialysis demand, hemodialysis schedule, hemodialysis time for each visited target patient, and urgency (priority) for each patient, or the like. Automatic matching between a target patient and a bed may be achieved through identifying the target patient.

According to an embodiment of the present disclosure, the patient information measurement device 10 may individually include an AI model, and may calculate the same-day hemodialysis demand for the target patient by using the AI model.

In other words, the patient information measurement device 10 may include the AI model. The patient information measurement device 10 may receive target patient information, may calculate the recommended same-day hemodialysis demand for the patient, and may deliver the calculated result to the server 40 and the hemodialysis device 30. In this case, the patient information measurement device 10 may transmit the calculated recommended same-day hemodialysis demand to the medical institution terminal 20 together with identification information and measurement health status information about the target patient and may receive the confirmation of a medical institution.

With regard to AI learning in this disclosure, the server 40 may train the target patient's health status data, such as the target patient's weight change data and blood pressure change, during a predetermined period and may predict the dry weight of the patient in this way. In this case, the server 40 may perform training with a training factor by labeling the ideal dry weight by using the type of medicine being taken, the dosage of the medicine, and the duration of the medicine as input data.

According to another embodiment, the dry weight prediction results of the target patient through AI learning may be used to correct the recommended hemodialysis demand calculated on the day of hemodialysis. For example, the recommended hemodialysis demand calculated on the day is based on health information (i.e. dry weight) measured on the day. The dry weight measured on the day may be corrected by using the AI-based dry weight prediction result of the target patient as a weight for the dry weight measured on the day, by comparing the dry weight measured on the day and the AI-based dry weight prediction results of the target patient. The corrected dry weight of the target patient may be used for the correction of the recommended hemodialysis demand of the target patient calculated on the day.

In the meantime, the server 40 may adjust the pre-calculated hemodialysis demand (e.g., reducing the pre-calculated hemodialysis demand by recalculating it) based on a health status event (e.g., a drop in blood pressure, headache, dizziness, or the like during hemodialysis) that occurs in previous hemodialysis records for each patient.

The server 40 may adjust the pre-calculated hemodialysis demand by recalculating it based on the dry weight correction content and the health status event.

With regard to the AI learning in this disclosure, general models such as LSTM of RNN technique, transformer, or the like may be used, but it is not limited thereto.

The server 40 may monitor data measurement results of vital signs (blood pressure, pulse, or body temperature) of a patient during hemodialysis and may detect or predict an anomaly during hemodialysis by using the AI model.

The hemodialysis device 30 may include at least one or more sensor devices. The hemodialysis device 30 may measure/collect real-time patient data such as the target patient's vital signs, blood pressure, blood flow rate, pulse, and vascular access path status through the sensor devices.

In particular, the hemodialysis device 30 may transmit/store the data thus measured and collected to a device management DB.

The server 40 may include the above-described AI model (an anomaly detection model and an event prediction model). The server 40 may detect an anomaly in a patient during hemodialysis by reflecting the patient's individual characteristics and the patient's vital signs collected in real time from the device management DB of the hemodialysis device 30 and may allow the medical institution terminal 20 and the hemodialysis device 30 to display related information on the displays of the medical institution terminal 20 and the hemodialysis device 30.

The anomaly detection model and the event prediction model may be included as a component of the processor 220.

The AI model according to an embodiment of the present disclosure may predict a hemodialysis patient's vital signs of the near future such as after 10 minutes or after 20 minutes, may detect an anomaly when the anomaly occur, and may recommend and provide corresponding actions. These corresponding actions may be recommended by labeling and training the corresponding actions in advance through the AI model. In the process, the blood pressure represents the patient's systemic blood pressure. The AI model may measure blood pressure, blood flow rate, pulse, body temperature, etc. in a vascular access path (the pressure of blood where a needle is inserted), may predict the degree of stenosis/occlusion of the vascular access path, and may predict the above-described content Oct. 20, 1930 minutes later.

With regard to the training of the AI model in the server 40, the patient's sequential data (see Table 1) collected from the sensor of the hemodialysis device 30 may be received in real time while the hemodialysis is in progress.

TABLE 1

| | | | Data | | |
|---|---|---|---|---|---|
| Dimension | Variable | Category | format | Unit | Descriptions |
| 1 | Age | | Floating | Years | Patient's age is |
| | | | point | of age | converted and display |
| | | | number | | into decimal points |
| | Gender | | Category | | Gender of patient, |
| | | | | | one-hot encoding as |
| | | | | | 1.0 for male/ 0.1 for |
| | | | | | female |
| 2 | | Male | | | |
| 3 | | Female | | | |
| 4 | Venous pressure | | Integer | mmHg | Venous pressure |
| | of vascular | | | | measured by sensor in |
| | access path | | | | hemodialysis device |
| 5 | Arterial pressure | | Integer | mmHg | Arterial pressure |
| | of vascular | | | | measured by sensor in |
| | access path | | | | hemodialysis device |
| 6 | Dialysis | | Integer | mmHg | Sensor automatically |
| | membrane | | | | measures pressure |
| | permeation | | | | difference between |
| | pressure | | | | blood side and |
| | | | | | hemodialysis liquid |
| | | | | | side of hemodialysis |
| | | | | | membrane |
| 7 | Ultrafiltration | | Integer | mmHg | Ratio at which water |
| | rate | | | | contained in blood is |
| | | | | | removed from body |
| | | | | | through pressure |
| 8 | Dialysis blood | | Integer | mmHg | Speed of blood flow |
| | flow rate | | | | entering hemodialysis |
| | | | | | device (maintaining |
| | | | | | blood flow at value set |
| | | | | | by medical staff |
| | | | | | through hemodialysis |
| | | | | | pump) |
| 9 | Dialysis | | Integer | mL/min | Perfusion rate of |
| | perfusion rate | | | | hemodialysis fluid |
| 10 | Diastolic blood | | Integer | mL/min | Patient's blood |
| | pressure | | | | pressure (diastolic) |
| 11 | Systolic blood | | Integer | mL/min | Patient's blood |
| | pressure | | | | pressure (systolic) |
| 12 | Pulse rate | | Integer | /min | Patient's pulse rate |
| 13 | Height | | Integer | cm | Patient's height |
| 14 | Dry weight | | Floating | kg | Patient's dry weight |
| | | | point | | set in advance by |
| | | | number | | medical staff |
| | | | | | (expected patient |
| | | | | | weight at end of |
| | | | | | dialysis targeted by |
| | | | | | medical staff) |
| 15 | Set dialysis | | Integer | min | Dialysis sample time |
| | treatment time | | | | (dialysis sample time |
| | | | | | is normally based on 4 |
| | | | | | hours. However, in |
| | | | | | practice, dialysis |
| | | | | | sample time is |
| | | | | | adjusted in range of 3 |
| | | | | | to 5 hours depending |
| | | | | | on patient's condition |
| | | | | | at each time) |
| 16 | Weight before | | Floating | kg | Patient's weight |
| | dialysis | | point | | immediately before |
| | | | number | | starting dialysis on |
| | | | | | same day |
| 17 | Weight after | | Floating | kg | Patient's weight |
| | dialysis | | point | | immediately after |
| | | | number | | completion of same- |
| | | | | | day dialysis |
| 18 | Total | | Integer | mL | Amount of fluid |
| | ultrafiltration | | | | (water) removed from |
| | amount | | | | patient's body by |
| | | | | | setting it on |
| | | | | | hemodialysis device |
| 19 | Cumulative | | Floating | mL | Cumulative amount of |
| | circulating | | point | | blood that has passed |
| | blood volume | | number | | out of body and |
| | | | | | through hemodialysis |
| | | | | | device during |
| | | | | | hemodialysis |
| | | | | | treatment |
| 20 | Surface area of | | Floating | m\hat{ }2 | Total surface area of |
| | dialysis | | point | | hemodialysis |
| | membrane | | number | | membrane used for |
| | | | | | hemodialysis |
| | | | | | (differing depending |
| | | | | | on type of |
| | | | | | hemodialysis |
| | | | | | membrane used) |
| | Types of | | | | |
| | dialysis | | | | |
| | membranes | | Category | | |
| 21 | | Revaclear | | | |
| | | 300 | | | |
| 22 | | Revaclear | | | |
| | | 400 | | | |
| 23 | | 170H | | | |
| 24 | | Theranova | | | |
| | | 400 | | | |
| | | | | | Method of connecting |
| | | | | | hemodialysis device |
| | Vascular access | | | | to patient's blood |
| | method | | Category | | vessels |
| 25 | | Arteriovenous | | | |
| | | fistula | | | |
| 26 | | Artificial | | | |
| | | blood | | | |
| | | vessel | | | |
| 27 | | Central | | | |
| | | venous | | | |
| | | catheter | | | |

As shown in Table 1, the hemodialysis device 30 may collect sequential data (e.g., about 27 pieces of data from 160 sensors in a hemodialysis device, such as blood flow, blood velocity, and pressure change), which is generated when the blood flow of the target patient passes through the hemodialysis device, and may transmit the sequential data to the server 40. However, the present disclosure is not limited to the content described in Table 1. The server 40 labels respective sequential data in Table 1 with a clinical event and may use the labeled result as learning data. Here, the clinical event may refer to an anomaly that occurs in a patient when specific sequential data occurs, such as a drop in blood pressure, chest pain, or headache.

The server 40 may generate the following prediction events. For example, the prediction events may be allowed to be displayed as acute (prediction of the possibility of stroke/cerebral hemorrhage when blood pressure rises sharply during the hemodialysis process, etc.), chronic (prediction of the possibility of future complications when the current state is maintained based on data measured during the hemodialysis process), or the like.

In other words, the server 40 may perform training by labeling the diagnosis, which is made by the medical staff to resolve the anomaly, with sequential data at a point in time when the anomaly occurs.

The operation of the AI model according to an embodiment of the present disclosure may be performed by including an anomaly prediction model and an anomaly determination model.

The anomaly determination model may apply several things simultaneously, and may use a model called “gradient boosting”, but is not limited thereto. For example, an RNN-based model (e.g., transformer) may be used as the anomaly determination model.

In the meantime, when a plurality of models are used, an average value calculating method or a voting method (a method of determining the output by majority vote on the results between a plurality of models) may be used as an output value calculating method, but is not limited thereto.

In signal transmission/reception between components constituting the AI-based hemodialysis data processing system 1 according to an embodiment of the present disclosure, encryption may be made by using RESTful API.

For example, when a hemodialysis device or a bed is placed at home, there is a need to remotely monitor whether the hemodialysis device is operating. To this end, Rest API may be used for encrypted communication between the hemodialysis device at home and a monitoring side (e.g., at least one of the medical institution terminal 20 and the server 40). When encrypted data is transmitted, a receiving side may decrypt and output the encrypted data. Moreover, data may be transmitted/received between a terminal and a server (a server, a nurse terminal, and a hemodialysis device) by using RESTful API. However, the encryption method is not limited to the one example, and a secure sockets layer (SSL) method using a web base may also be used.

Encrypted data may be transmitted from the hemodialysis device 30 to the device management DB. The device management DB may decrypt the encrypted data and may store the decrypted data as a file in a directory of its own DB. The device management DB may transmit the data to the server 40 in an encrypted state again.

It is also possible to detect whether there is an anomaly in a target patient, through a separate device for detecting status changes in the hemodialysis patient on a bed. In other words, the anomaly may be detected by detecting changes in the health status of the hemodialysis patient by using an auxiliary device during the hemodialysis process.

For example, the AI-based hemodialysis data processing system 1 may recognize a patient's face by using a camera as an example of the auxiliary device, may input facial expression changes as data, may predict the drop/rise in blood pressure from a change amount, and may determine the anomaly when the blood pressure falls/rises to be smaller than or equal to a threshold value. Alternatively, when the blood pressure changes rapidly to be greater than or equal to a predetermined value even though the blood pressure does not fall/rise to be greater/smaller than or equal to the threshold value, the AI-based hemodialysis data processing system 1 may determine anomaly detection.

A patient's pulse/movement data using a wearable device (e.g., a wrist ring, 3-axis gyro sensor, or the like) as another example of an auxiliary device may be used to detect an anomaly. For example, whether there is an anomaly in heart function or cardiovascular system (stroke, angina pectoris, or myocardial infarction) may be determined based on movement data such as pulse, oxygen saturation, and muscle spasms collected from the wearable device.

Furthermore, when an audio input device such as a microphone in a bed or a wearable device is used, the patient's audio data may be collected. Speech-to-text (STT) processing and natural language processing (NLP) may be perform on the collected audio data, and the processed result may also be used to detect an anomaly in the patient. The NLP-processed audio data may be directly transmitted to the medical institution terminal 20 and may be used as a reference for patient condition monitoring.

Besides, the anomaly may also be detected through the wearable device based on the patient's motion data, such as the patient falling from a bed. In case of the anomaly detection based on a patient's motion data, when there is the patient's movement that exceeds a threshold value, the anomaly may be detected or determined through comparison with the threshold value set based on the average value of movements of a plurality of patients during the hemodialysis process.

In the meantime, the above-described anomaly detecting or determining method may be appropriately combined.

As such, when the auxiliary device is used, the abnormality may be finally determined by using a method of mixing multiple results such as an average value or a majority vote method, by collecting and combining the anomaly detection results determined from the above-described AI model and the anomaly detection results determined from the auxiliary device. The final determination results may be output through the displays of the medical institution terminal 20 and the hemodialysis device 30.

In FIG. 5, an AI-based hemodialysis data processing method according to an embodiment of the present disclosure is described based on the processor 220 for convenience of description by the applicant, but is not limited thereto.

In step S101, the processor 220 may extract pre-stored identification information for a target patient.

In step S103, the processor 220 may obtain health status information measured for the target patient.

In step S105, the processor 220 may map the extracted identification information and the obtained health status information for the target patient.

In step S107, the processor 220 may acquire recommended same-day hemodialysis demand information calculated for the target patient based on the mapped information.

In step S109, the processor 220 may cause the mapped information and the obtained recommended same-day hemodialysis demand information for the target patient to be output.

In step S107, when acquiring the recommended same-day hemodialysis demand information, the processor 220 may predict dry weight data based on change data of a weight and blood pressure of the target patient during a predetermined period by using an AI model, may correct the predicted dry weight data by reflecting a result trained by labeling a standard dry weight based on a type, a dose, and duration of medication that the target patient is taking, and may calculate the recommended same-day hemodialysis demand based on the corrected dry weight data. In this case, when an event in which a health status of the target patient changes occurs in a previous hemodialysis process of the target patient, and a hemodialysis amount of the target patient is adjusted depending on the occurred event, the processor 220 may finally calculate data regarding the adjusted hemodialysis amount by correcting the recommended same-day hemodialysis demand based on the trained result.

The processor 220 may monitor a hemodialysis process of the target patient depending on the recommended same-day hemodialysis demand information that is finally calculated, and may acquire vital sign data during hemodialysis of the target patient in the monitoring process.

The vital sign data may include sequential data that comes out when a blood flow of the target patient passes through a hemodialysis device.

The processor 220 may perform training by labeling a clinical event in the respective sequential data by using an AI model.

Besides, the processor 220 may obtain vital sign prediction data according to the hemodialysis of the target patient based on the acquired vital sign data, may generate clinical event data according to the hemodialysis of the target patient based on the generated vital sign prediction data, and may cause the generated clinical event data to be output.

Also, the processor may determine whether there is a health anomaly according to the hemodialysis of the target patient based on at least one or more of vital sign data of the target patient, clinical event data, and unique characteristic information related to hemodialysis, and may cause the determination result of whether there is the health anomaly according to the hemodialysis of the target patient to be output.

The processor 220 may perform training in advance by labeling sequential data at a point in time, when the health anomaly occurs, and diagnosis and response content of a medical institution for resolving the health anomaly by using an AI model. The determination result of whether there is the health anomaly may include the diagnosis and the response content of the medical institution according to the trained result of the AI model.

The processor 220 may encrypt information and data obtained for the target patient by using Restful API.

According to at least one of various embodiments of the present disclosure described above, files created by a hemodialysis device may be automatically analyzed, and may be linked to or stored in an electronic medical record system. Accordingly, treatment efficiency may be improved because the patient's condition is determined in real time. Normal/abnormality may be determined by monitoring the target patient, and immediate response may be possible. Nursing records may be created by using a mobile device at the time of the nursing interview by medical staff right next to a patient receiving hemodialysis. Even without a uniform determination or a doctor's diagnosis, same-day hemodialysis demand, to which the patient's condition on that day is reflected, may be calculated by using the AI model within a patient information measurement device. Besides, a patient's anomaly may be predicted or detected during hemodialysis by using the AI model within the server, and relevant recommendation response information may be provided. When an event occurs, immediate response may be possible. It may be possible to add reliability to the AI model's anomaly detection/expected event prediction results by additionally using an auxiliary device.

Steps or operations of the method or algorithm described with regard to an embodiment of the present disclosure may be implemented directly in hardware, may be implemented with a software module executable by hardware, or may be implemented by a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or a computer-readable recording medium well known in the art to which the present disclosure pertains.

Although an embodiment of the present disclosure are described with reference to the accompanying drawings, it will be understood by those skilled in the art to which the present disclosure pertains that the present disclosure may be carried out in other detailed forms without changing the scope and spirit or the essential features of the present disclosure. Therefore, the embodiments described above are provided by way of example in all aspects, and should be construed not to be restrictive.

According to an embodiment of the present disclosure, AI-based customized hemodialysis information may be provided.

According to an embodiment of the present disclosure, events such as abnormal health status of a patient may be detected or predicted by monitoring a hemodialysis process based on AI, thereby preventing accidents or quickly and accurately responding to the accidents.

Effects of the present disclosure are not limited to the effects mentioned above, and other effects not mentioned will be clearly understood by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. An artificial intelligence (AI)-based hemodialysis data processing method performed by an electronic device, the method comprising:
extracting pre-stored identification information for a target patient;
acquiring health status information measured for the target patient;
mapping the extracted identification information and the acquired health status information;
acquiring recommended same-day hemodialysis demand information calculated for the target patient based on the mapped identification information and the mapped health status information; and
outputting the acquired recommended same-day hemodialysis demand information.

2. The method of claim 1, wherein the acquiring of the recommended same-day hemodialysis demand information includes:
predicting dry weight data based on change data of a weight and blood pressure of the target patient during a predetermined period by using an AI model;
correcting the predicted dry weight data by reflecting a result trained by labeling a standard dry weight based on a type, a dose, and duration of medication that the target patient is taking; and
calculating the recommended same-day hemodialysis demand based on the corrected dry weight data, and
wherein in the calculating of the recommended same-day hemodialysis demand, when an event in which a health status of the target patient changes occurs in a previous hemodialysis process of the target patient, and a hemodialysis amount of the target patient is adjusted depending on the occurred event, data regarding the adjusted hemodialysis amount is finally calculated by correcting the recommended same-day hemodialysis demand based on the trained result.

3. The method of claim 1, further comprising:
monitoring a hemodialysis process of the target patient depending on the recommended same-day hemodialysis demand information; and
acquiring vital sign data during hemodialysis of the target patient in the monitoring,
wherein the vital sign data includes respective sequential data that comes out when a blood flow of the target patient passes through a hemodialysis device.

4. The method of claim 3, further comprising:
performing training by labeling a clinical event in the respective sequential data by using an AI model.

5. The method of claim 3, further comprising:
obtaining data obtained by predicting a vital sign according to the hemodialysis of the target patient based on the acquired vital sign data;
generating clinical event data according to the hemodialysis of the target patient based on the predicted data; and
outputting the generated clinical event data.

6. The method of claim 3, further comprising:
determining whether there is a health anomaly according to the hemodialysis of the target patient based on the acquired vital sign data, clinical event data, and unique characteristic information related to hemodialysis; and
causing the determination result of whether there is the health anomaly to be output.

7. The method of claim 6, further comprising:
performing training by labeling sequential data at a point in time, when the health anomaly occurs, and diagnosis and response content of a medical institution for resolving the health anomaly by using an AI model,
wherein the determination result of whether there is the health anomaly includes the diagnosis and the response content of the medical institution according to the trained result of the AI model.

8. A computer-readable recording medium storing a program in combination with a computer being hardware to execute the AI-based hemodialysis data processing method of claim 1.

9. An AI-based hemodialysis data processing system, the system comprising:
at least one terminal; and
a server including a processor configured to perform data communication with the terminal,
wherein the processor is configured to:
extract pre-stored identification information for a target patient;
acquire health status information measured for the target patient;
map the extracted identification information and the acquired health status information;
acquire recommended same-day hemodialysis demand information calculated for the target patient based on the mapped identification information and the mapped health status information; and
cause the acquired recommended same-day hemodialysis demand information to be output.

10. The system of claim 9, wherein the processor is configured to:
when acquiring the recommended same-day hemodialysis demand information,
predict dry weight data based on change data of a weight and blood pressure of the target patient during a predetermined period by using an AI model;
correct the predicted dry weight data by reflecting a result trained by labeling a standard dry weight based on a type, a dose, and duration of medication that the target patient is taking;
calculate the recommended same-day hemodialysis demand based on the corrected dry weight data; and
when an event in which a health status of the target patient changes occurs in a previous hemodialysis process of the target patient, and a hemodialysis amount of the target patient is adjusted depending on the occurred event, finally calculate data regarding the adjusted hemodialysis amount by correcting the recommended same-day hemodialysis demand based on the trained result.

11. The system of claim 9, wherein the processor is configured to:
monitor a hemodialysis process of the target patient depending on the recommended same-day hemodialysis demand information; and
acquire vital sign data during hemodialysis of the target patient in the monitoring, and
wherein the vital sign data includes respective sequential data that comes out when a blood flow of the target patient passes through a hemodialysis device.

12. The system of claim 11, wherein the processor is configured to:
perform training by labeling a clinical event in the respective sequential data by using an AI model.

13. The system of claim 11, wherein the processor is configured to:
obtain data obtained by predicting a vital sign according to the hemodialysis of the target patient based on the acquired vital sign data;
generate clinical event data according to the hemodialysis of the target patient based on the predicted data; and
cause the generated clinical event data to be output.

14. The system of claim 11, wherein the processor is configured to:
determine whether there is a health anomaly according to the hemodialysis of the target patient based on the acquired vital sign data, clinical event data, and unique characteristic information related to hemodialysis; and
cause the determination result of whether there is the health anomaly to be output.

15. The system of claim 14, wherein the processor is configured to:
perform training by labeling sequential data at a point in time, when the health anomaly occurs, and diagnosis and response content of a medical institution for resolving the health anomaly by using an AI model, and
wherein the determination result of whether there is the health anomaly includes the diagnosis and the response content of the medical institution according to the trained result of the AI model.
